SEQUENCE LISTING (37 sequences)

SEQ ID NO: 1 - DNA, 21 bp, Homo sapiens
  CDS (1)..(21)
tacaatgagcccaaggtgacg

SEQ ID NO: 2 - protein, 7 aa, Homo sapiens
YNEPKVT

SEQ ID NO: 3 - DNA, 21 bp, Homo sapiens
  CDS (1)..(21)
agcctcccgcaacgcctggct

SEQ ID NO: 4 - protein, 7 aa, Homo sapiens
SLPQRLA

SEQ ID NO: 5 - DNA, 21 bp, Homo sapiens
  CDS (1)..(21)
gctggccgtgacctcaaacgc

SEQ ID NO: 6 - protein, 7 aa, Homo sapiens
AGRDLKR

SEQ ID NO: 7 - DNA, 21 bp, Rattus norvegicus
  CDS (1)..(21)
tacaatgagcccaaggtcaca

SEQ ID NO: 8 - protein, 7 aa, Rattus norvegicus
YNEPKVT

SEQ ID NO: 9 - DNA, 21 bp, Rattus norvegicus
  CDS (1)..(21)
aacctaccccaacgcctggca

SEQ ID NO: 10 - protein, 7 aa, Rattus norvegicus
NLPQRLA

SEQ ID NO: 11 - DNA, 21 bp, Rattus norvegicus
  CDS (1)..(21)
gcaggccgtgatctgaagcgc

SEQ ID NO: 12 - protein, 7 aa, Rattus norvegicus
AGRDLKR

SEQ ID NO: 13 - DNA, 24 bp, Homo sapiens
  CDS (1)..(24)
ctgcagaagttccgcggctcctcc

SEQ ID NO: 14 - protein, 8 aa, Homo sapiens
LQKFRGSS

SEQ ID NO: 15 - DNA, 24 bp, Rattus norvegicus
  CDS (1)..(24)
ctgcagaagttccgaggttcctca

SEQ ID NO: 16 - protein, 8 aa, Rattus norvegicus
LQKFRGSS

SEQ ID NO: 17 - DNA, 15 bp, Homo sapiens
  CDS (1)..(15)
aagttccgcggctcc

SEQ ID NO: 18 - protein, 5 aa, Homo sapiens
KFRGS

SEQ ID NO: 19 - DNA, 15 bp, Rattus norvegicus
  CDS (1)..(15)
aagttccgaggttcc

SEQ ID NO: 20 - protein, 5 aa, Rattus norvegicus
KFRGS

SEQ ID NO: 21 - DNA, 15 bp, Homo sapiens
  CDS (1)..(15)
cagaagttccgcggc

SEQ ID NO: 22 - protein, 5 aa, Homo sapiens
QKFRG

SEQ ID NO: 23 - DNA, 15 bp, Rattus norvegicus
  CDS (1)..(15)
cagaagttccgaggt

SEQ ID NO: 24 - protein, 5 aa, Rattus norvegicus
QKFRG

SEQ ID NO: 25 - DNA, 12 bp, Homo Sapiens
  CDS (1)..(12)
aagttccgcggc

SEQ ID NO: 26 - protein, 4 aa, Homo Sapiens
KFRG

SEQ ID NO: 27 - protein, 9 aa, Artificial
  Cysteines added to both termini of a protein with the amino acid sequence of SEQ ID NOs2 or 8 for cyclization.
CYNEPKVTC

SEQ ID NO: 28 - protein, 9 aa, Artificial
  Cysteines added to both termini of a protein with the amino acid sequence of SEQ ID NO4 for cyclization.
CSLPQRLAC

SEQ ID NO: 29 - protein, 9 aa, Artificial
  Cysteines added to both termini of a protein with the amino acid sequence of SEQ ID NO10 for cyclization.
CNLPQRLAC

SEQ ID NO: 30 - protein, 9 aa, Artificial
  Cysteines added to both termini of a protein with the amino acid sequence of SEQ ID NOs6 or 12 for cyclization.
CAGRDLKRC

SEQ ID NO: 31 - protein, 10 aa, Artificial
  Cysteines added to both termini of a protein with the amino acid sequence of SEQ ID NOs14 or 16 for cyclization.
CLQKFRGSSC

SEQ ID NO: 32 - protein, 7 aa, Artificial
  Cysteines added to both termini of a protein with the amino acid sequence of SEQ ID NOs18 or 20 for cyclization.
CKFRGSC

SEQ ID NO: 33 - protein, 7 aa, Artificial
  Cysteines added to both termini of a protein with the amino acid sequence of SEQ ID NOs22 or 24 for cyclization.
CQKFRGC

SEQ ID NO: 34 - protein, 6 aa, Artificial
  Cysteines added to both termini of a protein with the amino acid sequence of SEQ ID NO26 for cyclization.
CKFRGC

SEQ ID NO: 35 - protein, 8 aa, Artificial
  Peptide from nerve growth factor, with cysteines added to both termini for cyclization.
CTDKGKEC

SEQ ID NO: 36 - protein, 13 aa, Homo sapiens
LWAWLQKFRGSSS

SEQ ID NO: 37 - protein, 6 aa, Artificial
  Reverse sequence (retro-inverso) of shortened version of, e.g., SEQ ID NOs14 or 31, comprising D-amino acids in positions 1, 3, 4, 5, and 6 (s, r, f, k, and q, respectively), and cyclized by homodetic cyclization.  Can be represented as c[sGrfkq].
XGXXXX

(19) United States
(12) Patent Application Publication
(10) Pub. No.: US 2009/0131327 A1
(43) Pub. Date: May 21, 2009

(54) NOGO RECEPTOR FUNCTIONAL MOTIFS AND PEPTIDE MIMETICS RELATED THERETO AND METHODS OF USING THE SAME

(76) Inventors: Patrick Doherty, Middlesex (GB); Gareth Williams, Essex (GB)

Correspondence Address: FITZPATRICK CELLA HARPER & SCINTO, 30 ROCKEFELLER PLAZA, NEW YORK, NY, 10112, US

(21) Appl. No.: 11/913,032

(22) PCT Filed: Apr. 28, 2006
(86) PCT No.: PCT/US06/16217

§ 371 Date: Jun. 23, 2008

Related U.S. Application Data
(60) Provisional application No. 60/675,902, filed on Apr. 29, 2005.

(51) Int. Cl. A61K 38/08; C07K 7/00; G01N 33/566; C07K 16/18; A61K 38/07; C12N 5/06

(52) U.S. Cl. 514/16; 530/330; 530/328; 530/329; 530/317; 436/501; 435/375; 514/18; 514/17; 530/387.9

(57) ABSTRACT

The present invention provides novel isolated and purified polynucleotides and polypeptides related to functional motifs of the Nogo receptor 1 (NgR1) and use of peptides mimicking these functional motifs as antagonists to NgR1 ligands, e.g., myelin-associated glycoprotein, oligodendrocyte myelin glycoprotein, Nogo-A, Nogo-66, an antibody to Nogo receptor, an antibody to GT1b, an antibody to p75 neurotrophin receptor, and an antibody to Lingo-1, etc. The invention also provides antibodies to the mimetic peptide antagonists. The present invention is further directed to novel therapeutics and therapeutic targets and to methods of screening and assessing test compounds for treatments requiring axonal regeneration, i.e., reversal of the effects of NgR1 ligand binding to the NgR1 (i.e., producing inhibition of axonal growth). The present invention also is directed to novel methods for treating disorders arising from inhibition of axonal growth mediated by the binding of NgR1 ligands to the NgR1.

### RELATED APPLICATIONS

This application claims the benefit of priority from U.S. Provisional Patent Application No. 60/675,902, filed Apr. 29, 2005, which is hereby incorporated by reference herein in its entirety.

### BACKGROUND OF THE INVENTION

1. Field of the Invention

The invention relates to functional motifs of the Nogo receptor 1 (NgR1) and peptide mimetics related thereto, both of which may be used as antagonists to NgR1 ligands and, as such, may be useful in treating subjects in need of axonal regeneration (e.g., for antagonizing (e.g., reversing, decreasing, reducing, preventing, etc.) axonal growth inhibition mediated by such NgR1 ligands, and for screening for compounds that may also act as antagonists to NgR1 ligands to accomplish the reversal of such inhibition).

2. Related Background Art

The central nervous system shows very limited repair after injury; this has been postulated to be due, at least in part, to the presence of inhibitory products associated with damaged myelin that prevent axonal regeneration (Berry (1982) Bibl. Anat. 23:1-11). Early studies in this area identified two protein fractions from rat central myelin that contain inhibitory activity (Caroni and Schwab (1988) Neuron 1(1):85-96) and demonstrated that an antibody raised against these fractions could neutralize the nonpermissive substrate properties of central myelin (Caroni and Schwab (1988) J. Cell Biol. 106(4):1281-88). Furthermore, antibody delivery and immunotherapy strategies in animals have provided “proof-of-concept” evidence that some degree of regeneration within the damaged central nervous system can be obtained by counteracting the activity of the myelin inhibitors (Bregman et al. (1995) Nature 378:498-501; Schnell and Schwab (1990) Nature 343:269-72).

To date, three myelin molecules have been reported to be potent inhibitors of axonal growth: 1) the myelin-associated glycoprotein (MAG) (McKerracher et al. (1994) Neuron 13(4):805-11; Mukhopadhyay et al. (1994) Neuron 13(3):757-67), 2) Nogo (e.g., Nogo-A (e.g., the 66-residue extracellular domain of Nogo-A (Nogo-66))) (Chen et al. (2000) Nature 403:434-39; GrandPre et al. (2000) Nature 403:439-44; Prinjha et al. (2000) Nature 403:383-84) and 3) the oligodendrocyte myelin glycoprotein (Wang et al. (2002) Nature 417:941-44). A receptor complex in neurons containing the Nogo receptor 1 (NgR1) Domeniconi et al. (2002) Neuron 35(2):283-90; Fournier et al. (2001) Nature 409:341-46; Liu et al. (2002) Science 297:1190-93; Wang et al. (2002) Nature 420:74-78), the ganglioside GT1b (Collins et al. (1997) J. Biol. Chem. 272(2):1248-55; Vinson et al. (2001) J. Biol. Chem. 276(23):20280-85), the low affinity p75 neurotrophin receptor (p75NTR) (Wang et al. (2002) Nature 420:74-78; Wong et al. (2002) Nat. Neurosci. 5(12):1302-08), and Lingo-1 (Mi et al. (2004) Nat. Neurosci. 7(3):221-28), has been implicated in mediating the response to all three inhibitory molecules. Importantly, binding to the receptor complex is required for each inhibitor to mediate inhibitory activity.

Many studies point to the importance of the NgR1 as a potential therapeutic target (McGee and Strittmatter (2003) Trends Neurosci. 26(4):193-98). For example, the soluble ectodomain of the NgR1 can antagonize the inhibitory activity of myelin in a number of experimental paradigms (Fournier et al. (2002) J. Neurosci. 22(20):8876-83), and peptides derived from Nogo-A (e.g., a fragment of Nogo-66, e.g., NEP1-40) also promote axonal regeneration, presumably by binding to, but not activating, the receptor (GrandPre et al. (2002) Nature 417:547-51). The NgR1 has a prominent leucine-rich repeat (LRR) domain, which is composed of amino and carboxy terminal LRR modules that cap nine highly homologous LRR modules; two groups have recently resolved the crystal structure (Barton et al. (2003) EMBO J. 22(13):3291-302; He et al. (2003) Neuron 38(2):177-85). Deletion analysis studies suggest that the entire LRR domain of the receptor is important for the binding of Nogo-66, MAG and the NgR1 with itself.

Agents that interfere with the interaction of one or more NgR1 ligands (which may also be an axonal growth inhibitor(s)) with the NgR1 and/or the formation of the higher order receptor-signaling complex may have therapeutic potential and/or be useful biological tools, e.g., for antagonizing (e.g., reversing, decreasing, reducing, preventing, etc.) NgR1 ligand-mediated inhibition of axonal growth. In this context, if functional motifs could be identified on the NgR1, biologically active peptide mimetics could be developed as specific antagonists, or serve as useful tools in the drug discovery process (see generally, e.g., Hruby (2002) Nat. Rev. Drug Discov. 1(11):847-58). However, attempts to identify small functional motifs by conventional deletion mutagenesis are hampered because the overall “banana”-like shape of the structure of the NgR1 can easily be disrupted by mutations within the leucine-rich repeats. The present invention circumvents the problems encountered by deletion mutagenesis analysis, and identifies functional motifs of the NgR1. As such, the invention provides peptide mimetics as antagonists to NgR1 ligands (which are also axonal growth inhibitors), e.g., MAG, oligodendrocyte myelin glycoprotein, Nogo-A, etc. Active peptide mimetics, i.e., antagonistic drugs, may be therapeutic agents for a variety of conditions where axonal sprouting or long-range growth might restore function, e.g., a damaged central nervous system, e.g., due to a stroke, some other form of traumatic brain and/or spinal cord injury, etc. (see, e.g., Wiessner et al. (2003) J. Cereb. Blood Flow Metab. 23(2):154-65; Moon and Bunge (2005) J. Neurol. Phys. Ther. 29:55-69).

### SUMMARY OF THE INVENTION

The present invention is based on the identification of functional motifs within the Nogo receptor 1 (NgR1). The invention is also based on the use of peptides mimicking such functional motifs to antagonize NgR1 ligands (NgR1L), which are also axonal growth inhibitors (e.g., myelin-associated glycoprotein, oligodendrocyte myelin glycoprotein, Nogo-A, Nogo-66, an antibody to Nogo receptor, an antibody to GT1b, an antibody to p75 neurotrophin receptor, and an antibody to Lingo-1, etc.). In one embodiment, a putative and/or actual functional motif of the NgR1 has and/or consists essentially of an amino acid sequence selected from the group consisting of YNEPKVT (SEQ ID NOs:2 and 8), LQKFRGSS (SEQ ID NOs:14 and 16), SLPQRLA (SEQ ID NO:4), NLPQRLA (SEQ ID NO:10) and AGRDLKR (SEQ ID NOs:6 and 12). In another embodiment of the invention, a peptide mimetic of a putative and/or actual functional motif of the NgR1 of the invention is provided as an antagonist to one or more NgR1 ligand(s) (NgR1L), i.e., an antagonist to at least one NgR1L. For example, the invention provides an antagonist to an NgR1L (i.e., an antagonist to at least one NgR1L) comprising a polypeptide comprising an amino acid sequence selected from the group consisting of the amino acid sequence of YNEPKVT (SEQ ID NOs:2 and 8), LQKFRGSS (SEQ ID NOs:14 and 16), SLPQRLA (SEQ ID NO:4), NLPQRLA (SEQ ID NO:10), AGRDLKR (SEQ ID NOs:6 and 12), and the amino acid sequences of active fragments thereof.

In one embodiment, the invention provides an antagonist to an NgR1 ligand comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. In several embodiments of the invention, an antagonist to an NgR1 ligand comprises a polypeptide comprising an amino acid sequence selected from the group consisting of the amino acid sequences LQKFRGSS (SEQ ID NOs:14 and 16), KFRGS (SEQ ID NOs:18 and 20), and QKFRG (SEQ ID NOs:22 and 24). In other embodiments, an antagonist of the invention is acetylated and/or amide blocked. In other embodiments, an antagonist of the invention is cyclized (e.g., via homodetic cyclization or a disulfide bond). For example, in one embodiment, the invention provides an antagonist to an NgR1L comprising a polypeptide comprising the amino acid sequence KFRG (SEQ ID NO:26), wherein the polypeptide is cyclized, e.g., by homodetic cyclization, which is a form of cyclization in which the ring consists solely of amino acid residues in eupeptide linkage. In another embodiment, the antagonist comprises at least one D-amino acid. In another embodiment, the antagonist comprises the amino acid sequence of SGRFKQ (SEQ ID NO:37; alternate representation of an antagonist of the invention comprising a homodetic cyclic polypeptide (c[ ]) comprising the amino acid sequence of SEQ ID NO:37 with D-type normative amino acids (lower case letters), i.e.: c[sGrfkq]), or an active fragment(s) thereof.

In other embodiments, an antagonist of the invention is cyclized by means of a disulfide bond. In one embodiment, the invention provides a cyclized antagonist to an NgR1 ligand comprising a polypeptide comprising an amino acid sequence selected from the group consisting of the amino acid sequence of SEQ ID NO:31, the amino acid sequence of SEQ ID NO:32, the amino acid sequence of SEQ ID NO:33, the amino acid sequence of SEQ ID NO:34, and the amino acid sequences of active fragments thereof. In one embodiment, the invention provides an antagonist of at least one NgR1 ligand comprising a polypeptide comprising the amino acid sequence of CLQKFRGSSC (SEQ ID NO:31). In another embodiment, the antagonist comprises a polypeptide comprising the amino acid sequence of CKFRGSC (SEQ ID NO:32). In another embodiment, the antagonist comprises a polypeptide comprising the amino acid sequence of CQKFRGC (SEQ ID NO:33). In another embodiment, the antagonist comprises a polypeptide comprising the amino acid sequence of CKFRGC (SEQ ID NO:34). In several embodiments, an antagonist of the invention comprises at least one D-amino acid. In other embodiments, an antagonist of the invention is acetylated and/or amide blocked. In another embodiment, the antagonists described above antagonize an NgR1 binding fragment of an NgR1 ligand selected from the group consisting of myelin-associated glycoprotein, oligodendrocyte myelin glycoprotein, Nogo-A, Nogo-66, an antibody to Nogo receptor, an antibody to GT1b, an antibody to p75 neurotrophin receptor, and an antibody to Lingo-1.

The invention also provides methods of using the antagonists of the invention, e.g., methods of screening for other antagonists (e.g., test compounds), and methods of antagonizing NgR1 ligand-mediated inhibition of axonal growth in a sample or subject (e.g., a human subject). In one embodiment, the invention provides a method of screening for compounds that antagonize NgR1 ligands comprising the steps of contacting a sample containing an NgR1 ligand and an antagonist of the invention with the compound; and determining whether the interaction between the NgR1 ligand and the antagonist of the invention in the sample is decreased relative to the interaction of the NgR1 ligand and the antagonist of the invention in a sample not contacted with the compound, whereby a decrease in the interaction of the NgR1 ligand and the antagonist of the invention in the sample contacted with the compound identifies the compound as one that competes with the antagonist of the invention. In some embodiments of these methods, the antagonist comprises a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. Additionally, in some embodiments, the compound is further identified as one that antagonizes at least one NgR1 ligand.

The invention also provides a method of antagonizing inhibition of axonal growth mediated by an NgR1 ligand in a sample comprising the step of contacting the sample with an antagonist of the invention. In one embodiment, the antagonist to the at least one NgR1 ligand is a peptide that mimics a functional motif of the NgR1. The invention also provides a method of antagonizing inhibition of axonal growth in a sample comprising the step of contacting the sample with an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. In several embodiments, the inhibition of axonal growth is mediated by at least one NgR1 ligand. Additionally, in some embodiments, the antagonizing of inhibition of axonal growth results in regeneration of axons.

In one embodiment, the invention provides a method of regenerating axons and/or antagonizing inhibition of axonal growth in a subject (e.g., a human subject) comprising administering to the subject an antagonist of the invention. For example, the invention provides a method of antagonizing inhibition of axonal growth in a subject comprising the step of administering to the subject an effective amount of an antagonist to at least one NgR1 ligand, e.g., wherein the antagonist to the at least one NgR1 ligand is a peptide that mimics a functional motif of the NgR1. In another embodiment, the invention provides a method of antagonizing inhibition of axonal growth in a subject comprising the step of administering to the subject an effective amount of an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof. In several embodiments, the inhibition of axonal growth is mediated by at least one NgR1 ligand. In other embodiments, the antagonizing of inhibition of axonal growth results in regeneration of axons. In another embodiment, the method of regenerating axons and/or antagonizing inhibition of axonal growth in a subject comprises administering to the subject an antagonist of the invention, wherein the subject has suffered an injury to the central nervous system, e.g., wherein the subject has suffered from a stroke and/or some other form of traumatic brain and/or spinal cord injury, etc. In another embodiment, the subject suffers from, or has suffered from, a neuronal degenerative disease, e.g., multiple sclerosis, Parkinson's disease, Alzheimer's disease, etc.

In addition, the present invention provides pharmaceutical compositions comprising an antagonist of the invention, and routes of administration of such a composition, for use in the methods of the invention. In some embodiments, a pharmaceutical composition of the invention comprises a pharmaceutically acceptable carrier and an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

The invention also provides an antagonist to an NgR1 ligand comprising a polypeptide comprising an amino acid sequence selected from the group consisting of the amino acid sequence of SEQ ID NO:2, the amino acid sequence of SEQ ID NO:4, the amino acid sequence of SEQ ID NO:6, the amino acid sequence of SEQ ID NO:10, and the amino acid sequences of active fragments thereof. In some embodiments, the polypeptide is cyclized (e.g. via a disulfide bond, etc.).

The invention also provides an isolated antibody capable of specifically binding to a polypeptide comprising an amino acid sequence selected from the group consisting of the amino acid sequences of SEQ ID NOs:2, 4, 6, 8, 10, 12, 14, 16, 18, 20, 22, 24, 26, 27, 28, 29, 30, 31, 32, 33, 34, 37, and the amino acid sequences of active fragments thereof. In some embodiments, the antibody is produced in response to an immunogen comprising an antagonist to at least one NgR1 ligand. Also provided is an isolated antibody capable of specifically binding to an antagonist to at least one NgR1 ligand.

The present invention also provides kits comprising an antagonist of the invention to aid in practicing the methods disclosed herein.

### BRIEF DESCRIPTION OF THE DRAWINGS

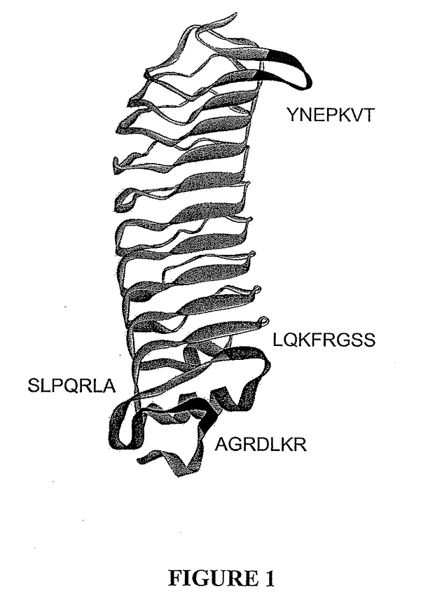

A ribbon diagram of the Nogo receptor 1 (NgR1), showing the four putative and/or actual functional motifs, is shown in FIG. 1.

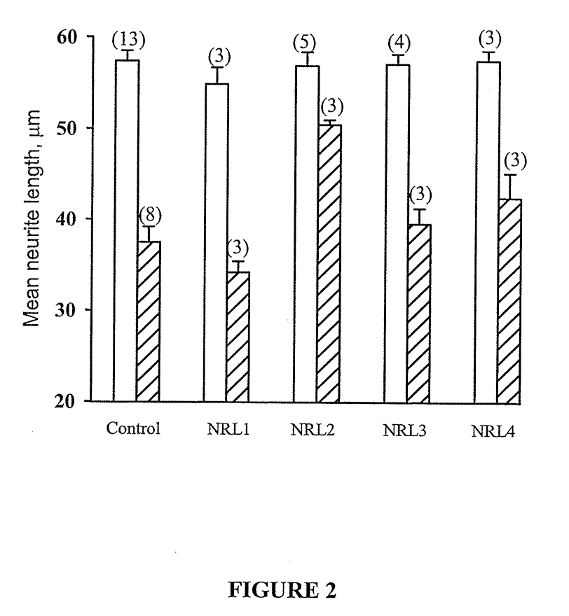

Results from between 3 and 13 independent experiments [as noted in the parentheses] were pooled to obtain the mean length of the longest cerebellar neurite (μm; y-axis)±SEM (bars) from 100-120 neurons cultured over monolayers of established 3T3 cells in media supplemented for 23-27 hr without MAG-Fc (white columns) or with MAG-Fc at 20-25 μg/ml (cross-hatched columns) in the absence (control) or presence of 100 μg/ml NRL peptides 1-4 (x-axis), as shown in FIG. 2.

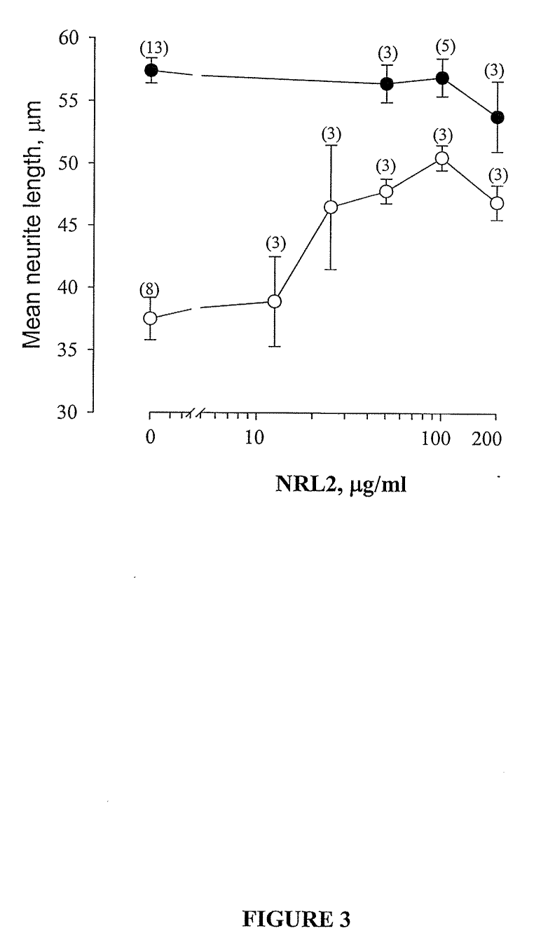

Results from between 3 and 13 independent experiments [as noted in the parentheses] were pooled to obtain the mean length of the longest cerebellar neurite (μm; y-axis)±SEM (bars) from 120-150 neurons cultured over monolayers of established 3T3 cells in control media (filled circles) or media supplemented with the MAG-Fc at 25 μg/ml (open circles) in the presence of the artificially cyclized, acetylated, and amide-blocked NRL2 peptide (N-Ac-CLQKFRGSSC-NH₂ (SEQ ID NO:31)) at the given concentrations (x-axis), as shown in FIG. 3.

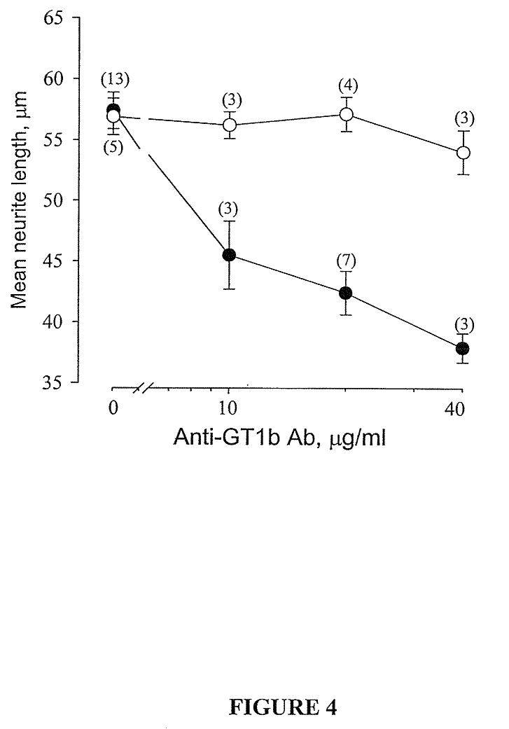

Results from between 3 and 13 independent experiments [as noted in the parentheses] were pooled to obtain the mean length of the longest cerebellar neurite (μm; y-axis)±SEM (bars) from 100-120 neurons cultured over monolayers of established 3T3 cells in media containing 0-40 μg/ml anti-GT1b antibody in the absence (filled circles) or presence (open circles) of the NRL2 peptide (N-Ac-CLQKFRGSSC-NH₂ (SEQ ID NO:31)) at 100 μg/ml, as shown in FIG. 4.

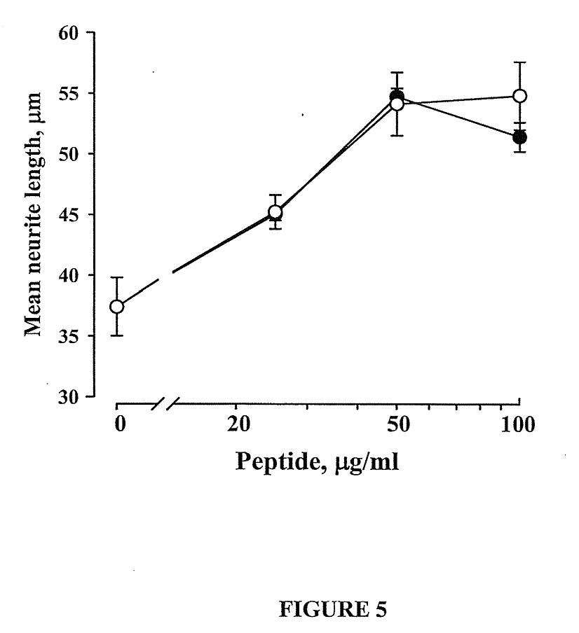

The mean lengths of the longest neurite (μm; y-axis)±SEM (bars) from about 100-120 neurons of 3 to 5 independent cultures of cerebellar neurons over monolayers of established 3T3 cells in media supplemented with 20 μg/ml MAG-Fc alone (0 μg/ml peptide) or in the presence of increasing concentrations (μg/ml; x-axis) of NRL2a (N-Ac-CKFRGSC-NH₂ (SEQ ID NO:32); filled circles) or NRL2b (N-Ac-CQKFRGC-NH₂ (SEQ ID NO:33); open circles) are shown in FIG. 5.

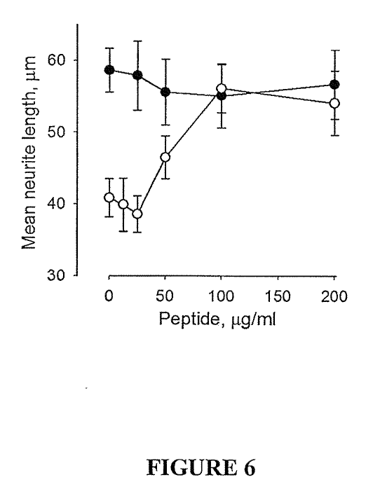

The mean lengths of the longest neurite (μm; y-axis)±SEM (bars) from about 100-120 neurons of 2 independent cultures of cerebellar neurons over monolayers of established 3T3 cells in control media (filled circles) or media supplemented with 20 μg/ml MAG-Fc (open circles), both with increasing concentrations (μg/ml; x-axis) of hriNRL2 (N-Ac c[sGrfkq]-NH₂ (SEQ ID NO:37)) are shown in FIG. 6.

### DETAILED DESCRIPTION OF THE INVENTION

The limitations presented by conventional deletion analysis were overcome by adopting a rational approach to identify putative and/or actual functional motifs in the Nogo receptor 1 (NgR1) (see Example 2.1). Based on this approach, three independent small-constrained peptides that mimic an exposed loop at the carboxy terminal region of the LRR structure of the NgR1 were identified. These peptides can act as antagonists to NgR1 ligands, (e.g., myelin-associated glycoprotein, oligodendrocyte myelin glycoprotein, Nogo-A, Nogo-66, an antibody to Nogo receptor, an antibody to GT1b, an antibody to p75 neurotrophin receptor, and an antibody to Lingo-1), i.e., can act to antagonize (e.g., reverse, decrease, reduce, prevent, etc.) the biological consequences of an NgR1 ligand(s) binding to the NgR1 complex in neurons (e.g., inhibition of axonal growth (Examples 2.2-2.4) and/or the formation of the higher order receptor-signaling complex). As such, the invention provides polynucleotides and polypeptides related to the putative and/or actual functional motifs and/or mimetic peptide antagonists.

### Polynucleotides and Polypeptides

The present invention provides novel isolated and purified polynucleotides and polypeptides homologous to putative and/or actual functional domains of the Nogo receptor 1 (NgR1). It is part of the invention that peptide mimetics to putative and/or actual functional domains of the NgR1 may be used as antagonists to NgR1 ligands, i.e., to inhibit the biological effect of NgR1 ligand binding to the NgR1.

For example, the invention provides purified and isolated polynucleotides encoding three putative NgR1 functional motifs, which may function as NgR1 ligand antagonists, herein designated “NRL1,” “NRL3,” and “NRL4.” Preferred DNA sequences of the invention include genomic and cDNA sequences and chemically synthesized DNA sequences.

The nucleotide sequences of cDNAs encoding human NRL1 (hNRL1), human NRL3 (hNRL3), and human NRL4 (hNRL4), designated human cDNA, are set forth in SEQ ID NOs:1, 3, and 5, respectively. Polynucleotides of the present invention also include polynucleotides that hybridize under stringent conditions to SEQ ID NOs:1, 3, or 5, or complements thereof, and/or encode polypeptides that retain substantial biological activity of hNRL1, hNRL3, or hNRL4, respectively. Polynucleotides of the present invention also include continuous portions of the sequences set forth in SEQ ID NOs:1, 3, or 5 comprising at least 12 consecutive nucleotides.

The amino acid sequences of hNRL1, hNRL3, and hNRL4 are set forth in SEQ ID NOs:2, 4, and 6, respectively. Polypeptides of the present invention also include continuous portions of any of the sequences set forth in SEQ ID NOs:2, 4, and 6, comprising at least 4 consecutive amino acids. Polypeptides of the invention also include any of the sequences set forth in SEQ ID NOs:2, 4, and 6, including continuous portions thereof, wherein one or more of the L-amino acids are replaced with their corresponding D-amino acids. Polypeptides of the present invention also include any continuous portion of any of the sequences set forth in SEQ ID NO:2, 4, and 6 that retains substantial biological activity (i.e., an active fragment) of full-length human hNRL1, hNRL3, and hNRL4, respectively. Additionally, a polypeptide of the invention may be acetylated and/or amide blocked using well-known methods. Polynucleotides of the present invention also include, in addition to those polynucleotides of human origin described above, polynucleotides that encode any of the amino acid sequences set forth in SEQ ID NO:2, 4, or 6, or continuous portions thereof (e.g., active fragments thereof), and that differ from the polynucleotides of human origin described above only due to the well-known degeneracy of the genetic code.

The nucleotide sequences of cDNAs encoding rat NRL1 (rNRL1), rat NRL3 (rNRL3), and rat NRL4 (rNRL4), designated rat cDNA, are set forth in SEQ ID NOs:7, 9, and 11, respectively. Polynucleotides of the present invention also include polynucleotides that hybridize under stringent conditions to SEQ ID NOs:7, 9, or, 11, or complements thereof, and/or encode polypeptides that retain substantial biological activity of rNRL1, rNRL3, or rNRL4, respectively. Polynucleotides of the present invention also include continuous portions of the sequences set forth in SEQ ID NOs:7, 9, or 11 comprising at least 12 consecutive nucleotides.

The amino acid sequences of rNRL1, rNRL3, and rNRL4 are set forth in SEQ ID NOs:8, 10, and 12, respectively. Polypeptides of the present invention also include continuous portions of any of the sequences set forth in SEQ ID NOs:8, 10, and 12, comprising at least 4 consecutive amino acids. Polypeptides of the invention also include any of the sequences set forth in SEQ ID NOs:8, 10, and 12, including continuous portions thereof, wherein one or more of the L-amino acids are replaced with their corresponding D-amino acids. Polypeptides of the present invention also include any continuous portion of any of the sequences set forth in SEQ ID NOs:8, 10, and 12 that retains substantial biological activity (i.e., an active fragment) of full-length rNRL1, rNRL3, and rNRL4, respectively. Additionally, a polypeptide of the invention may be acetylated and/or amide blocked using well-known methods. Polynucleotides of the present invention also include, in addition to those polynucleotides of rat origin described above, polynucleotides that encode any of the amino acid sequences set forth in SEQ ID NOs:8, 10, and 12, or continuous portions thereof (e.g., active fragments thereof), and that differ from the polynucleotides of rat origin described above only due to the well-known degeneracy of the genetic code.

The invention also provides purified and isolated polynucleotides encoding a novel NgR1 functional motif, which may also be used as a mimetic peptide antagonist to an NgR1 ligand, herein designated “NRL2.” Preferred DNA sequences of the invention include genomic and cDNA sequences and chemically synthesized DNA sequences.

The nucleotide sequence of a cDNA encoding human NRL2 (hNRL2), designated human cDNA, is set forth in SEQ ID NO:13. Polynucleotides of the present invention also include polynucleotides that hybridize under stringent conditions to SEQ ID NO:13, or its complement, and/or encode polypeptides that retain substantial biological activity of hNRL2. Polynucleotides of the present invention also include continuous portions of the sequence set forth in SEQ ID NO:13 comprising at least 12 consecutive nucleotides.

The amino acid sequence of hNRL2 is set forth in SEQ ID NO:14. Polypeptides of the present invention also include continuous portions of the sequence set forth in SEQ ID NO:14 comprising at least 4 consecutive amino acids. Polypeptides of the invention also include the sequence set forth in SEQ ID NO:14, including continuous portions thereof, wherein one or more of the L-amino acids are replaced with their corresponding D-amino acids. Polypeptides of the present invention also include any continuous portion of the sequence set forth in SEQ ID NO:14 that retains substantial biological activity (i.e., an active fragment) of full-length hNRL2, e.g., KFRG (i.e., SEQ ID NO:26). Additionally, a polypeptide of the invention may be acetylated and/or amide blocked using well-known methods. Polynucleotides of the present invention also include, in addition to those polynucleotides of human origin described above, polynucleotides that encode the amino acid sequence set forth in SEQ ID NO:14 or a continuous portion thereof (e.g., an active fragment thereof), and that differ from the polynucleotides of human origin described above only due to the well-known degeneracy of the genetic code.

The nucleotide sequence of a cDNA encoding rat NRL2 (rNRL2), designated rat cDNA, is set forth in SEQ ID NO:15. Polynucleotides of the present invention also include polynucleotides that hybridize under stringent conditions to SEQ ID NO:15, or its complement, and/or encode polypeptides that retain substantial biological activity of rNRL2. Polynucleotides of the present invention also include continuous portions of the sequence set forth in SEQ ID NO:15 comprising at least 12 consecutive nucleotides.

The amino acid sequence of rNRL2 is set forth in SEQ ID NO:16. Polypeptides of the present invention also include continuous portions of the sequence set forth in SEQ ID NO:16 comprising at least 4 consecutive amino acids. Polypeptides of the invention also include the sequence set forth in SEQ ID NO:16, including continuous portions thereof, wherein one or more of the L-amino acids are replaced with their corresponding D-amino acids. Polypeptides of the present invention also include any continuous portion of the sequence set forth in SEQ ID NO:16 that retains substantial biological activity (i.e., an active fragment) of full-length rNRL2, e.g., KFRG (i.e., SEQ ID NO:26). Additionally, a polypeptide of the invention may be acetylated and/or amide blocked using well-known methods. Polynucleotides of the present invention also include, in addition to those polynucleotides of rat origin described above, polynucleotides that encode the amino acid sequence set forth in SEQ ID NO:16 or a continuous portion thereof (e.g., an active fragment thereof), and that differ from the polynucleotides of rat origin described above only due to the well-known degeneracy of the genetic code.

The invention also provides purified and isolated polynucleotides encoding a novel mimetic peptide antagonist to an NgR1 ligand, herein designated “NRL2a.” Preferred DNA sequences of the invention include genomic and cDNA sequences and chemically synthesized DNA sequences.

The nucleotide sequence of a cDNA encoding human NRL2a (hNRL2a), designated human cDNA, is set forth in SEQ ID NO:17. Polynucleotides of the present invention also include polynucleotides that hybridize under stringent conditions to SEQ ID NO:17, or its complement, and/or encode polypeptides that retain substantial biological activity of hNRL2a. Polynucleotides of the present invention also include continuous portions of the sequence set forth in SEQ ID NO:17 comprising at least 12 consecutive nucleotides.

The amino acid sequence of hNRL2a is set forth in SEQ ID NO:18. Polypeptides of the present invention also include continuous portions of the sequence set forth in SEQ ID NO:18 comprising at least 4 consecutive amino acids. Polypeptides of the invention also include the sequence set forth in SEQ ID NO:18, including continuous portions thereof, wherein one or more of the L-amino acids are replaced with their corresponding D-amino acids. Polypeptides of the present invention also include any continuous portion of the sequence set forth in SEQ ID NO:18 that retains substantial biological activity (i.e., an active fragment) of full-length hNRL2a, e.g., KFRG (SEQ ID NO:26). Additionally, a polypeptide of the invention may be acetylated and/or amide blocked using well-known methods. Polynucleotides of the present invention also include, in addition to those polynucleotides of human origin described above, polynucleotides that encode the amino acid sequence set forth in SEQ ID NO:18 or a continuous portion thereof (e.g., an active fragment thereof), and that differ from the polynucleotides of human origin described above only due to the well-known degeneracy of the genetic code.

The nucleotide sequence of a cDNA encoding rat NRL2a (rNRL2a), designated rat cDNA, is set forth in SEQ ID NO:19. Polynucleotides of the present invention also include polynucleotides that hybridize under stringent conditions to SEQ ID NO:19, or its complement, and/or encode polypeptides that retain substantial biological activity of rNRL2a. Polynucleotides of the present invention also include continuous portions of the sequence set forth in SEQ ID NO:19 comprising at least 12 consecutive nucleotides.

The amino acid sequence of rNRL2a is set forth in SEQ ID NO:20. Polypeptides of the present invention also include continuous portions of the sequence set forth in SEQ ID NO:20 comprising at least 4 consecutive amino acids. Polypeptides of the invention also include the sequence set forth in SEQ ID NO:20, including continuous portions thereof, wherein one or more of the L-amino acids are replaced with their corresponding D-amino acids. Polypeptides of the present invention also include any continuous portion of the sequence set forth in SEQ ID NO:20 that retains substantial biological activity (i.e., an active fragment) of full-length rNRL2a, e.g., KFRG (SEQ ID NO:26). Additionally, a polypeptide of the invention may be acetylated and/or amide blocked using well-known methods. Polynucleotides of the present invention also include, in addition to those polynucleotides of rat origin described above, polynucleotides that encode the amino acid sequence set forth in SEQ ID NO:20 or a continuous portion thereof, and that differ from the polynucleotides of rat origin described above only due to the well-known degeneracy of the genetic code.

The invention also provides purified and isolated polynucleotides encoding another novel mimetic peptide antagonist to an NgR1 ligand, herein designated “NRL2b.” Preferred DNA sequences of the invention include genomic and cDNA sequences and chemically synthesized DNA sequences.

The nucleotide sequence of a cDNA encoding human NRL2b (hNRL2b), designated human cDNA, is set forth in SEQ ID NO:21. Polynucleotides of the present invention also include polynucleotides that hybridize under stringent conditions to SEQ ID NO:21, or its complement, and/or encode polypeptides that retain substantial biological activity of hNRL2b. Polynucleotides of the present invention also include continuous portions of the sequence set forth in SEQ ID NO:21 comprising at least 12 consecutive nucleotides.

The amino acid sequence of hNRL2b is set forth in SEQ ID NO:22. Polypeptides of the present invention also include continuous portions of the sequence set forth in SEQ ID NO:22 comprising at least 4 consecutive amino acids. Polypeptides of the invention also include the sequence set forth in SEQ ID NO:22, including continuous portions thereof, wherein one or more of the L-amino acids are replaced with their corresponding D-amino acids. Polypeptides of the present invention also include any continuous portion of the sequence set forth in SEQ ID NO:22 that retains substantial biological activity (i.e., an active fragment) of full-length hNRL2b, e.g., KFRG (SEQ ID NO:26). Additionally, a polypeptide of the invention may be acetylated and/or amide blocked using well-known methods. Polynucleotides of the present invention also include, in addition to those polynucleotides of human origin described above, polynucleotides that encode the amino acid sequence set forth in SEQ ID NO:22 or a continuous portion thereof, and that differ from the polynucleotides of human origin described above only due to the well-known degeneracy of the genetic code.

The nucleotide sequence of a cDNA encoding rat NRL2b (rNRL2b), designated rat cDNA, is set forth in SEQ ID NO:23. Polynucleotides of the present invention also include polynucleotides that hybridize under stringent conditions to SEQ ID NO:23, or its complement, and/or encode polypeptides that retain substantial biological activity of rNRL2b. Polynucleotides of the present invention also include continuous portions of the sequence set forth in SEQ ID NO:23 comprising at least 12 consecutive nucleotides.

The amino acid sequence of rNRL2b is set forth in SEQ ID NO:24. Polypeptides of the present invention also include continuous portions of the sequence set forth in SEQ ID NO:24 comprising at least 4 consecutive amino acids. Polypeptides of the invention also include the sequence set forth in SEQ ID NO:24, including continuous portions thereof, wherein one or more of the L-amino acids are replaced with their corresponding D-amino acids. Polypeptides of the present invention also include any continuous portion of the sequence set forth in SEQ ID NO:24 that retains substantial biological activity (i.e., an active fragment) of full-length rNRL2b, e.g., KFRG (SEQ ID NO:26). Additionally, a polypeptide of the invention may be acetylated and/or amide blocked using well-known methods. Polynucleotides of the present invention also include, in addition to those polynucleotides of rat origin described above, polynucleotides that encode the amino acid sequence set forth in SEQ ID NO:24 or a continuous portion thereof, and that differ from the polynucleotides of rat origin described above only due to the well-known degeneracy of the genetic code.

The invention also provides purified and isolated polynucleotides encoding the novel NgR1 functional motifs and the mimetic peptide antagonists of the invention, e.g., NRL2, NRL2a, and NRL2b, as cyclized mimetic peptides. Preferred DNA sequences of the invention include genomic and cDNA sequences and chemically synthesized DNA sequences. One of skill in the art will recognize that the present invention also includes other cyclized molecules, such as cyclized mimetic peptides based on NRL1, NRL3, and NRL4, etc. Additionally, a polypeptide of the invention may be acetylated and/or amide blocked using well-known methods.

For example, the amino acid sequences of artificially cyclized, acetylated and amide blocked NRL2, NRL2a, and NRL2b are set forth in SEQ ID NOs:31, 32, and 33, respectively. Polypeptides of the present invention also include continuous portions of any of the sequences set forth in SEQ ID NOs:31, 32, or 33, comprising at least 4 consecutive amino acids. Polypeptides of the present invention also include any continuous portion of any of the sequences set forth in SEQ ID NOs:31, 32, or 33 that retains substantial biological activity (i.e., an active fragment) of full-length NRL2, NRL2a, or NRL2b, respectively, e.g., KFRG (SEQ ID NO:26). Another polypeptide of the invention is the artificially cyclized, acetylated, and amide blocked KFRG (SEQ ID NO:34). As other examples, the amino acid sequences of artificially cyclized, acetylated and amide blocked NRL1 (human or rat), human NRL3, rat NRL3, and NRL4 (human or rat) are set forth in SEQ ID NOs:27, 28, 29, and 30, respectively. Polypeptides of the invention also include any of the sequences set forth in SEQ ID NOs:27, 28, 29, 30, 31, 32, 33, or 34, including continuous portions thereof, wherein one or more of the L-amino acids are replaced with their corresponding D-amino acids.

Based on the amino acid sequences provided in SEQ ID NOs:27, 28, 29, 30, 31, 32, 33, or 34, a skilled artisan could determine one or more DNA sequences that would encode for each of such peptides. As such, polynucleotides of the present invention also include polynucleotides (e.g., genomic, cDNA, and chemically synthesized sequences) that encode an amino acid sequence set forth in SEQ ID NOs:27, 28, 29, 30, 31, 32, 33, or 34, or continuous portions thereof.

For example, a nucleotide sequence of that encodes KFRG, is set forth in SEQ ID NO:25. Polynucleotides of the present invention also include polynucleotides that hybridize under stringent conditions to SEQ ID NO:25, or its complement, and/or encode polypeptides that retain substantial biological activity of KFRG. Polynucleotides of the present invention also include continuous portions of the sequence set forth in SEQ ID NO:25 comprising at least 9 consecutive nucleotides.

As described above, the amino acid sequence of KFRG is set forth in SEQ ID NO:26. Polypeptides of the present invention also include continuous portions of the sequence set forth in SEQ ID NO:26 comprising at least 3 consecutive amino acids. Polypeptides of the invention also include the sequence set forth in SEQ ID NO:26, including continuous portions thereof, wherein one or more of the L-amino acids are replaced with their corresponding D-amino acids. Polypeptides of the present invention also include any continuous portion of the sequence set forth in SEQ ID NO:26 that retains substantial biological activity (i.e., an active fragment) of full-length human KFRG, e.g., KFR. Additionally, a polypeptide of the invention may be cyclized, acetylated and/or amide blocked using well-known methods. Polynucleotides of the present invention also include, in addition to those polynucleotides described above, polynucleotides that encode the amino acid sequence set forth in SEQ ID NO:26 or a continuous portion thereof (e.g., an active fragment thereof), and that differ from the polynucleotides described above only due to the well-known degeneracy of the genetic code.

The isolated polynucleotides of the present invention may be used as hybridization probes and primers to identify and isolate nucleic acids having sequences identical to, or similar to, those encoding the disclosed polynucleotides. Hybridization methods for identifying and isolated nucleic acids include polymerase chain reaction (PCR), Southern hybridization, and Northern hybridization, and are well known to those skilled in the art.

Hybridization reactions can be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | Poly- | Hybrid | Hybridization | Wash |
|---|---|---|---|---|
| Stringency | nucleotide | Length | Temperature and | Temperature |
| Condition | Hybrid | (bp)¹ | Buffer² | and Buffer² |
| A | DNA:DNA | >50 | 65° C.; 1X SSC | 65° C.; |
| | | | -or- | 0.3X SSC |
| | | | 42° C.; 1X SSC, | |
| | | | 50% formamide | |
| B | DNA:DNA | <50 | T_{B}*; 1X SSC | T_{B}*; 1X SSC |
| C | DNA:RNA | >50 | 67° C.; 1X SSC | 67° C.; |
| | | | -or- | 0.3X SSC |
| | | | 45° C.; 1X SSC, | |
| | | | 50% formamide | |
| D | DNA:RNA | <50 | T_{D}*; 1X SSC | T_{D}*; 1X SSC |
| E | RNA:RNA | >50 | 70° C.; 1X SSC | 70° C.; |
| | | | -or- | 0.3x SSC |
| | | | 50° C.; 1X SSC, | |
| | | | 50% formamide | |
| F | RNA:RNA | <50 | T_{F}*; 1X SSC | T_{f}*; 1X SSC |
| G | DNA:DNA | >50 | 65° C.; 4X SSC | 65° C.; |
| | | | -or- | 1X SSC |
| | | | 42° C.; 4X SSC, | |
| | | | 50% formamide | |
| H | DNA:DNA | <50 | T_{H}*; 4X SSC | T_{H}*; 4X SSC |
| I | DNA:RNA | >50 | 67° C.; 4X SSC | 67° C.; |
| | | | -or- | 1X SSC |
| | | | 45° C.; 4X SSC, | |
| | | | 50% formamide | |
| J | DNA:RNA | <50 | T_{J}*; 4X SSC | T_{J}*; 4X SSC |
| K | RNA:RNA | >50 | 70° C.; 4X SSC | 67° C.; |
| | | | -or- | 1X SSC |
| | | | 50° C.; 4X SSC, | |
| | | | 50% formamide | |
| L | RNA:RNA | <50 | T_{L}*; 2X SSC | T_{L}*; 2X SSC |
| M | DNA:DNA | >50 | 50° C.; 4X SSC | 50° C.; |
| | | | -or- | 2X SSC |
| | | | 40° C.; 6X SSC, | |
| | | | 50% formamide | |
| N | DNA:DNA | <50 | T_{N}*; 6X SSC | T_{N}*; 6X SSC |
| O | DNA:RNA | >50 | 55° C.; 4X SSC | 55° C.; |
| | | | -or- | 2X SSC |
| | | | 42° C.; 6X SSC, | |
| | | | 50% formamide | |
| P | DNA:RNA | <50 | T_{P}*; 6X SSC | T_{P}*; 6X SSC |
| Q | RNA:RNA | >50 | 60° C.; 4X SSC | 60° C.; |
| | | | -or- | 2X SSC |
| | | | 45° C.; 6X SSC, | |
| | | | 50% formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) may be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ (°C.) = 2(# of A + T bases) + 4(# of G + C bases). For hybrids between 18 and 49 base pairs in length, Tₘ(° C.) = 81.5 + 16.6(log₁₀NA⁺) + 0.41(% G + C) − (600/N), where N is the number of bases in the hybrid, and Na⁺ is the concentration of sodium ions in the hybridization buffer (Na⁺ for 1xSSC = 0.165M).
Additional examples of stringency conditions for polynucleotide hybridization are provided in Sambrook et al. (1989) Molecular Cloning: A Laboratory Manual, Chs. 9 &11, Cold Spring Harbor Laboratory Press, Cold Spring Harbor, NY, and Ausubel et al., eds. (1995) Current Protocols in Molecular Biology, Sects. 2.10 &6.3-6.4, John Wiley &Sons, Inc., herein incorporated by reference.

The isolated polynucleotides of the present invention may also be used as hybridization probes and primers to identify and isolate DNAs having sequences encoding polypeptides homologous to the disclosed polynucleotides. These homologs are polynucleotides and polypeptides isolated from species different than those of the disclosed polypeptides and polynucleotides, or within the same species, but with significant sequence similarity to the disclosed polynucleotides and polypeptides. Preferably, polynucleotide homologs have at least 60% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

The isolated polynucleotides of the present invention may also be used as hybridization probes and primers to identify cells and tissues that express the polypeptides of the present invention and the conditions under which they are expressed.

The isolated polynucleotides of the present invention may be operably linked to an expression control sequence such as the pMT2 and pED expression vectors for recombinant production of the polypeptides of the present invention. General methods of expressing recombinant proteins are well known in the art.

A number of cell types may act as suitable host cells for recombinant expression of the polypeptides of the present invention. Mammalian host cells include, e.g., COS cells, CHO cells, 293 cells, A431 cells, 3T3 cells, CV-1 cells, HeLa cells, L cells, BHK21 cells, HL-60 cells, U937 cells, HaK cells, Jurkat cells, normal diploid cells, cell strains derived from in vitro culture of primary tissue, and primary explants.

Alternatively, it may be possible to recombinantly produce the polypeptides of the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides of the present invention are made in yeast or bacteria, it may be necessary to modify them by, e.g., phosphorylation or glycosylation of appropriate sites, in order to obtain functionality. Such covalent attachments may be accomplished using well-known chemical or enzymatic methods.

The polypeptides of the present invention may also be recombinantly produced by operably linking the isolated polynucleotides of the present invention to suitable control sequences in one or more insect expression vectors, such as baculovirus vectors, and employing an insect cell expression system. Materials and Methods for baculoviris/Sf9 expression systems are commercially available in kit form (e.g., the MaxBac® kit, Invitrogen, Carlsbad, Calif.).

Following recombinant expression in the appropriate host cells, the polypeptides of the present invention may then be purified from culture medium or cell extracts using known purification processes, such as gel filtration and ion exchange chromatography. Purification may also include affinity chromatography with agents known to bind the polypeptides of the present invention. These purification processes may also be used to purify the polypeptides of the present invention from natural sources.

Alternatively, the polypeptides of the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen (Carlsbad, Calif.), respectively. The polypeptides of the present invention can also be tagged with a small epitope and subsequently identified or purified using a specific antibody to the epitope. A preferred epitope is the FLAG epitope, which is commercially available from Eastman Kodak (New Haven, Conn.).

The polypeptides of the present invention may also be produced by known conventional chemical synthesis. Methods for chemically synthesizing the polypeptides of the present invention are well known to those skilled in the art. Such chemically synthetic polypeptides may possess biological properties in common with the natural, purified polypeptides, and thus may be employed as biologically active or immunological substitutes for the natural polypeptides.

The polypeptides of the present invention also encompass molecules that are structurally different from the disclosed polypeptides (e.g., which have a slightly altered sequence), but which have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques and kits for such alterations, substitutions, replacements, insertions, or deletions are well known to those skilled in the art.

### Antibodies

Antibody molecules capable of specifically binding to the polypeptides of the present invention may be produced by methods well known to those skilled in the art. For example, monoclonal antibodies can be produced by generation of hybridomas in accordance with known methods. Hybridomas formed in this manner are then screened using standard methods, such as enzyme-linked immunosorbent assay (ELISA), to identify one or more hybridomas that produce an antibody that specifically binds with the polypeptides of the present invention.

A full-length polypeptide of the present invention may be used as the immunogen, or, alternatively, antigenic peptide fragments of the polypeptides may be used. For example, the immunogen may be a functional motif of the NgR1 (e.g., one or more of the amino acid sequences of SEQ ID NOs:2, 4, 6, 8, 10, 12, 14, and 16) and/or a related peptide or cyclized peptide (e.g., one or more of the amino acid sequences of SEQ ID NOs:18, 20, 22, 24, 26, 27, 28, 29, 30, 31, 32, 33, 34, and 37). An antigenic peptide of a polypeptide of the present invention comprises at least four continuous amino acid residues and encompasses an epitope such that an antibody raised against the peptide forms a specific immune complex with the polypeptide. Preferably, the antigenic peptide comprises at least four amino acid residues, more preferably at least seven amino acid residues, and even more preferably at least nine amino acid residues.

As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with a polypeptide of the present invention to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody display libraries can be found in the literature.

Polyclonal sera and antibodies may be produced by immunizing a suitable subject with a polypeptide of the present invention. The antibody titer in the immunized subject may be monitored over time by standard techniques, such as with ELISA using immobilized marker protein. If desired, the antibody molecules directed against a polypeptide of the present invention may be isolated from the subject or culture media and further purified by well known techniques, such as protein A chromatography, to obtain an IgG fraction.

Fragments of antibodies to the polypeptides of the present invention may be produced by cleavage of the antibodies in accordance with methods well known in the art. For example, immunologically active F(ab′) and F(ab′)₂ fragments may be generated by treating the antibodies with an enzyme such as pepsin.

Additionally, chimeric, humanized, and single-chain antibodies to the polypeptides of the present invention, comprising both human and nonhuman portions, may be produced using standard recombinant DNA techniques. Humanized antibodies may also be produced using transgenic mice that are incapable of expressing endogenous immunoglobulin heavy and light chain genes, but that can express human heavy and light chain genes.

### Screening Assays and Sources of Test Compounds

The polynucleotides and polypeptides of the present invention may also be used in screening assays to identify pharmacological agents or lead compounds for other antagonists to NgR1 ligands, which may be used to antagonize (e.g., reverse, decrease, reduce, prevent, etc.) NgR1L-mediated inhibition of axonal growth. For example, samples containing an antagonist of the invention, e.g., a polypeptide comprising an amino acid sequence selected from the group consisting of SEQ ID NOs:2, 4, 6, 10, 14, 18, 22, and 26-34, and an NgR1 ligand (including an NgR1 binding fragment of an NgR1 ligand (e.g., NEP1-40)) can be contacted with one of a plurality of test compounds (e.g., small organic molecules or biological agents), and the interaction in each of the treated samples can be compared to the interaction of the antagonist of the invention and an NgR1 ligand in untreated samples or in samples contacted with different test compounds to determine whether any of the test compounds provides a substantially decreased level of antagonist:NgR1 ligand interactions. In a preferred embodiment, the identification of test compounds capable of modulating the activity of antagonist:NgR1 ligand interactions is performed using high-throughput screening assays, such as provided by BIACORE® (Biacore International AB, Uppsala, Sweden), BRET (bioluminescence resonance energy transfer), and FRET (fluorescence resonance energy transfer) assays, as well as ELISA. One of skill in the art will recognize that test compounds capable of decreasing levels of antagonist:NgR1 ligand interactions may be antagonists of NgR1L (e.g., because they bind to NgR1L and block NgR1:NgR1L interactions) or agonists of NgR1L (e.g., because they bind to, e.g., KFRG and activate inhibition of axonal growth). Such antagonistic or agonistic test compounds screened in the above-described manner may then be further distinguished, e.g., tested for their ability to antagonize NgR1L-mediated axonal growth inhibition, or to enhance NgR1L-mediated axonal growth inhibition, respectively, using well-known methods, e.g., the neurite outgrowth assay described in Example 1.1.

The test compounds of the present invention may be obtained from a number of sources. For example, combinatorial libraries of molecules are available for screening. Using such libraries, thousands of molecules can be screened for inhibitory activity. Preparation and screening of compounds can be screened as described above or by other methods well known to those of skill in the art. The compounds thus identified can serve as conventional “lead compounds” or can be used as the actual therapeutics.

### Methods of Treatment

Peptide mimetics related to functional motifs of the NgR1, particularly peptides comprising the amino acid sequence of KFRG, may be used as antagonists to the axonal growth inhibition effects of NgR1 ligands, e.g., myelin-associated glycoprotein, oligodendrocyte myelin glycoprotein, Nogo-A, Nogo-66, an antibody to Nogo receptor, an antibody to GT1b, an antibody to p75 neurotrophin receptor, and an antibody to Lingo-1. As such, the present invention provides both prophylactic and therapeutic methods for treatments requiring axonal regeneration, i.e., antagonism (e.g., reversal, decrease, reduction, prevention, etc.) of axonal growth inhibition, that involve administration of an antagonist of the invention. A skilled artisan will recognize that such methods of treatment will be particularly useful in subjects who may suffer from, or who suffer from, or who may have suffered from, a brain injury caused by, e.g., stroke, multiple sclerosis, Parkinson's disease, Alzheimer's disease, etc. The methods involve contacting cells (either in vitro, in vivo, or ex vivo) with an antagonist of the invention in an amount effective to antagonize (e.g., reverse, decrease, reduce, prevent, etc.) the activity of NgR1 ligands, e.g., the biological consequences of one or more NgR1 ligands binding to the NgR1 complex in neurons (e.g., the inhibition of axonal growth and/or the formation of the higher order receptor-signaling complex). The antagonist may be any molecule that antagonizes the activity of NgR1 ligands, including, but not limited to, small molecules and peptide inhibitors.

For example, small molecules (usually organic small molecules) that antagonize the activity of NgR1 ligands (e.g., myelin-associated glycoprotein, oligodendrocyte myelin glycoprotein, Nogo-A, Nogo-66, an antibody to Nogo receptor, an antibody to GT1b, an antibody to p75 neurotrophin receptor, and an antibody to Lingo-1) may be used to, e.g., reverse NgR1 ligand-mediated axonal growth inhibition. Novel antagonistic small molecules may be identified by the screening methods described above, and may be used in the treatment methods of the present invention described here.

Decreased activity of NgR1 ligands in an organism in need of axonal regeneration but afflicted with (or at risk for) inhibition of axonal growth mediated by NgR1 ligands, or in an involved cell from such an organism, may also be achieved using peptide inhibitors, e.g., the mimetic peptide antagonists of the invention, that bind to and inhibit the activity of NgR1 ligands. Peptide inhibitors include peptide pseudosubstrates that prevent NgR1 ligands from interacting with the NgR1. Peptide inhibitors that antagonize, or may antagonize, NgR1 ligands are disclosed herein as mimetic peptide antagonists, and include, but are not limited to, KFRG (SEQ ID NO:26), LQKFRGSS (SEQ ID NOs:14 and 16), KFRGS (SEQ ID NOs:18 and 20), and QKFRG (SEQ ID NO:22 and 24). In some embodiments, these peptide inhibitors are cyclized via disulfide bonds (e.g., SEQ ID NOs:31, 32, 33, and 34) to improve the ability of the peptides to act as antagonists (see Williams et al. (2000) J. Biol. Chem. 275(6):4007-12; Williams et al. (2000)Mol. Cell. Neurosci. 15(5):456-64). Cyclized and noncyclized NgR1 ligand peptide inhibitors may be chemically synthesized. Additionally, the peptide inhibitors of the invention may be acetylated and/or amide blocked using well-known methods. One can provide a cell (e.g., a neuron) with a peptide inhibitor in vitro, in vivo, or ex vivo using the techniques described below.

### Administration

Any of the compounds described herein (preferably a mimetic peptide or small molecule antagonist of the invention) can be administered in vivo in the form of a pharmaceutical composition for treatments requiring antagonism of axonal growth inhibition, i.e., axonal regeneration. The pharmaceutical composition may be administered by any number of routes, including, but not limited to, oral, nasal, intraventricular, rectal, topical, sublingual, subcutaneous, intravenous, intramuscular, intraarterial, intramedullary, intrathecal, intraperitoneal, intraarticular, or transdermal routes. In addition to the active ingredients, the pharmaceutical composition(s) may contain a pharmaceutically acceptable carrier(s). Such compositions may contain, in addition to any of the compounds described herein and an acceptable carrier(s), various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

For any compound, the therapeutically effective dose can be estimated initially either in cell culture or in animal models. The therapeutically effective dose refers to the amount of active ingredient that ameliorates the condition or its symptoms. Therapeutic efficacy and toxicity in cell cultures or animal models may be determined by standard pharmaceutical procedures (e.g., ED₅₀: the dose therapeutically effective in 50% of the population; LD₅₀: the dose lethal to 50% of the population). The dose ratio between therapeutic and toxic effects is the therapeutic index, and can be expressed as the ratio ED₅₀/LD₅₀. Pharmaceutical compositions that exhibit large therapeutic indexes are preferred.

The data obtained from cell culture and animal models can then be used to formulate a range of dosages for the compound for use in mammals, preferably humans. The dosage of such a compound preferably lies within a range of concentrations that includes the ED₅₀ with little to no toxicity. The dosage may vary within this range depending upon the composition form employed and the administration route utilized.

Another aspect of the present invention relates to kits for carrying out the administration of NgR1 ligand antagonists (e.g., the peptide mimetic antagonists of the invention), either alone or with another therapeutic compound(s) or agent(s). In one embodiment, the kit comprises one or more NgR1 ligand antagonists formulated with a pharmaceutically acceptable carrier(s).

The entire contents of all references, patents, and published patent applications cited throughout this application are hereby incorporated by reference herein.

### EXAMPLES

The following Examples provide illustrative embodiments of the invention and do not in any way limit the invention. One of ordinary skill in the art will recognize that numerous other embodiments are encompassed within the scope of the invention.

### Example 1

### Materials and Methods

### Example 1.1

### Neurite Outgrowth Assays

Cerebellar neurons isolated from postnatal day 2/3 rat pups were cultured over monolayers of 3T3 cells (Doherty et al. (1991) Neuron 6(2):247-58) essentially as previously described (Williams et al. (1994) Neuron 13(3):583-94). Monolayers were established by seeding ˜80,000 cells into individual chambers of an eight-chamber tissue culture slide coated with poly-L-lysine and fibronectin. The cell lines, and monolayers, were maintained in Dulbecco's modified Eagle's medium supplemented with 10% fetal calf serum (FCS). Cocultures were established by removing the media from the monolayers and seeding ˜6000 dissociated cerebellar neurons into each well in SATO medium (modified from Doherty et al. (1990) Neuron 5(2):209-19; Dulbecco's modified Eagle's medium supplemented with 2% FBS, 33% bovine albumin, 0.62 μg/ml progesterone, 161 μg/ml putrescine, 4 μg/ml L-thyroxine, 0.387 μg/ml selenium, and 3.37 μg/ml tri-iodo-thyronine (components from Sigma-Aldrich, St. Louis, Mo.)). Monolayers were established for 24 hours prior to addition of the neurons and the cultures were maintained for ˜23-27 hr. Following careful fixation with 4% paraformaldehyde, the neurons were immunostained with a GAP-43 antibody, and the mean length of the longest neurite per cell was measured for ˜120-150 neurons, again as previously described (Williams et al. (1994) Neuron 13(3):583-94).

### Example 1.2

### Structures

For the purposes of molecular modeling, the 1M10 (pdb accession number) glycoprotein Ib alpha in complex with von Willebrand factor (Huizinga et al. (2002) Science 297:1176-79) and the 1OZN (pdb accession number) structure of the NgR1 (He et al. (2003) Neuron 38(2):177-85) were used. Swiss PDB software packages were used to isolate the structure of various motifs from the binding interfaces of the crystals, and Accelrys software was used to generate images.

### Example 1.3

### Reagents

Synthetic peptides were all obtained from a commercial supplier (Multiple Peptide Systems, San Diego, Calif.). All peptides were purified to the highest grade by reverse-phase HPLC and obtained at the highest level of purity (>97%). With all peptides, there was no indication of higher molecular weight species. Where peptide sequences are underlined, this denotes a peptide that has been cyclized via a disulfide bond between the given cysteine residues. All peptides were acetylated (e.g., denoted with “N—Ac—”) and amide blocked (e.g., denoted with “—NH₂”). Recombinant MAG-Fc chimera was obtained from R&D Systems (Minneapolis, Minn.) and used at final concentrations ranging from 5-25 μg/ml. The monoclonal antibody to GT1b (clone GMR5) was obtained from Seikagaku America (Falmouth, Mass.) and was used at a final concentration of 20 μg/ml. All reagents were diluted into the coculture media and, in general, added to the cultures just prior to the plating of the neurons.

### Example 2

### Results

### Example 2.1

### Design of NgR1Loop Peptides

The structure of the NgR1 has been resolved (Barton et al. (2003) EMBO J. 22(13):3291-302; He et al. (2003) Neuron 38(2):177-85), but not as a component of a ligand/receptor complex. However, proteins with leucine-rich repeat (LRR) domains might use an evolutionarily conserved mechanism to engage ligands, and functional motifs in one receptor might be deduced from the identification of functional motifs in another receptor. Based on this hypothesis, the public domain for crystal structures of LRR molecules with their ligands was searched. One such structure is of the glycoprotein Ib alpha in complex with von Willebrand factor (pdb accession 1M10) (Huizinga et al. (2002) Science 297:1176-79). Although the NgR1 has one extra LRR motif relative to glycoprotein Ib alpha, the two structures are quite similar (not shown). In glycoprotein Ib alpha, the N and C terminal exposed loops are crucial to the interaction with the ligand. Based on this analysis, the equivalent loops and a number of putative functional motifs on the NgR1 were hypothesized, as shown in FIG. 1.

### Example 2.2

### Effects of Four NgR1 Loop Peptides on Neurite Outgrowth

Peptide mimetics of binding motifs in proteins often function as antagonists in biological assays, particularly if they are constrained by a disulfide bond (see, e.g., Williams et al. (2000) J. Biol. Chem. 275(6):4007-12; Williams et al. (2000) Mol. Cell. Neurosci. 15(5):456-64). Based on this, cyclic peptide mimetics of the four putative and/or actual motifs on the NgR1 that are highlighted in FIG. 1 were designed. These peptides were coded

| | |
|---|---|
| NRL1 (N-Ac-CYNEPKVTC-NH₂), | (SEQ ID NO:27) |
| NRL2 (N-Ac-CLQKFRGSSC-NH₂), | |
| NRL3 (N-Ac-CSLPQRLAC-NH₂) | |
| and | |
| NRL4 (N-Ac-CAGRDLKRC-NH₂). | |

MAG was the first inhibitory component of myelin to be identified based on its ability to inhibit neurite outgrowth from postnatal rat cerebellar neurons (Mukhopadhyay et al. (1994) Neuron 13(3):757-67). It can also inhibit neurite outgrowth when presented to neurons as a soluble Fc chimera (Tang et al. (1997) Mol. Cell. Neurosci. 9:333-46). NgR1 function is required for MAG inhibition of neurite outgrowth (Domeniconi et al. (2002) Neuron 35(2):283-90; Liu et al. (2002) Science 297:1190-93). Consequently, in order to determine if the mimetic peptides could antagonize, (e.g., reverse, decrease, reduce, prevent, etc.) NgR1 function (e.g., reverse NgR1-ligand-mediated inhibition of axonal growth), the peptides were tested for their ability to antagonize MAG-mediated inhibition of axonal growth. Postnatal day 2/3 cerebellar neurons were cultured over monolayers of 3T3 fibroblasts for ˜23-27 hr; under these conditions the MAG-Fc inhibited neurite outgrowth in these samples in a dose-dependent manner (not shown) with a robust inhibition seen at 20 μg/ml (FIG. 2). The ability of the NRL peptides to antagonize MAG-mediated inhibition of axonal growth was tested in a number of independent experiments. None of the peptides significantly inhibited neurite outgrowth in control (i.e., without MAG-Fc) media, and consequently, the peptides do not appear to have nonspecific effects on neuronal viability or function (FIG. 2). In control (i.e., without NRL peptides) media, MAG-Fc (20 μg/ml) substantially inhibited neurite outgrowth (FIG. 2). Likewise, in the presence of NRL1, NRL3 or NRL4 peptides at 100 μg/ml, MAG-Fc also substantially inhibited neurite outgrowth (FIG. 2). However, the inhibitory activity of the MAG-Fc was largely antagonized (i.e., reversed, overcome, prevented, etc.) by the presence of the NRL2 peptide (FIG. 2). In order to evaluate the efficacy of the NRL2 peptide, the ability of various concentrations of NRL2 to overcome the inhibitory activity of 25 μg/ml of the soluble MAG-Fc chimera was tested. Results obtained from at least three independent experiments have been pooled to generate FIG. 3. These results confirm that NRL2 has little effect on control (i.e., without MAG-Fc) neurite outgrowth when tested at up to 200 μg/ml. The results also show that the ability of the peptide to reverse the MAG-Fc-mediated inhibition of axonal growth is dose-dependent, and plateaus at 50 μg/ml (˜45 μM).

### Example 2.3

### NRL2 Inhibits the Function of a GT1b Antibody

The ganglioside GT1b appears to be part of the NgR1 complex that transmits inhibitory signals to neurons (Yamashita et al. (2002) J. Cell. Biol. 157(4):565-70) and accordingly, an antibody to GT1b can inhibit neurite outgrowth in a manner similar to the MAG-Fc (Vinson et al. (2001) J. Biol. Chem. 276(23):20280-85). An anti-GT1b antibody inhibited neurite outgrowth in a dose-dependent manner (FIG. 4). The inhibitory effects of anti-GT1b were also reversed in the presence of the 100 μg/ml of the NRL2 peptide, even when anti-GT1b was added at up to 40 μg/ml. These data confirm that the effects of the GT1b antibody are specific (in that they can be antagonized by a small peptide), and demonstrate that the NRL2 peptide can antagonize activation of the NgR1 complex by two independent ligands, i.e., can reverse NgR1 ligand-mediated inhibition of axonal growth.

### Example 2.4

### Identification of Key Functional Amino Acids in the NRL2 Sequence

Structural analyses of the NgR1 show that the most conspicuous amino acids within the NRL2 peptide sequence are the positively charged lysine (K) and arginine (R); both are highly solvent exposed, with their side chains clearly available for binding (data not shown). Of the surrounding amino acids, the phenylalanine (F) is buried in the structure, but might play a role in stabilizing the local region. The glycine and serine are partially solvent exposed, but look less likely as candidates to mediate a binding interaction. Based on this analysis, two small peptides that both have the key lysine and arginine within them were designed. These were NRL2a (N-Ac-CKFRGSC-NH₂ (SEQ ID NO:32)) and NRL2b (N-Ac-COKFRGC-NH₂ (SEQ ID NO:33)) peptides; note that these peptides contain a common four amino acid motif from the NgR1 loop sequence (KFRG (SEQ ID NO:26)). Both peptides had no effect on neurite outgrowth in control (i.e., without MAG-Fc) media (not shown); their ability to antagonize NgR1-ligand-mediated inhibition of axonal growth, i.e., to “promote” growth in the presence of the MAG-Fc, is shown in FIG. 5. Basal neurite outgrowth in control media was 57.4±1.1 μm (n=13) and this was reduced to 37.5±1.7 μm (n=8) in the presence of the MAG-Fc (20 μg/ml) (FIG. 5). Within the inhibitory environment, both peptides “promoted” neurite outgrowth, with significant effects seen at 25 μg/ml (30 μM) and maximal effects seen at 50 μg/ml (60 μM). At this higher concentration, the inhibitory activity of the MAG-Fc was effectively antagonized (i.e., decreased, reduced, abolished, prevented, etc.). This suggests that the functional activity within the NRL2 sequence resides within the KFRG motif (and, in fact, perhaps within the KFR motif).

### Example 2.5

### A Homodetic Retro-Inverso Mimetic Peptide Antagonist Based on NRL2

To increase the potency and in vivo stability of a potential NgR antagonist, a homodetic retro-inverso mimetic peptide (hriNRL2; SEQ ID NO:37), based on NRL2, was constructed. The hriNRL2 mimetic peptide was similar to NRL2 except for the following: 1) it did not comprise terminal cysteines, which are not part of the parent Nogo receptor sequence, 2) it was cyclized through a more stable peptide bond, referred to as homodetic cyclization, 3) it did not comprise the leucine at position 2 and the serine at position 9 of the NRL2 sequence, because NRL2a (Ac-CKFRGSC-NH₂ (SEQ ID NO:32)) and NRL2b (Ac-CQKFRGC-NH₂ (SEQ ID NO:33)) proved to be as effective as NRL2 in antagonizing MAG inhibition, 4) its L-type amino acids were replaced by their chiral partners, specifically, by normative D-type amino acids, and 5) its sequence was reversed to ensure that the side chain orientations were preserved. Consequently, the sequence of hriNRL2 peptide is c[sGrfiq], where c[ ] refers to homodetic cyclization and the lower case letters refer to D-type amino acids. Note that glycine (G) has no chirality as it has no side chain. FIG. 6 demonstrates the ability of hriNRL2 to antagonize NgR1 ligand-mediated inhibition of axonal growth, particularly, to reverse MAG-mediated inhibition of neurite outgrowth over 3T3 cells.

### Example 3

### Discussion

Until the present studies, no known small binding motifs had been identified in the NgR1. However, LRR proteins might use an evolutionarily conserved mechanism to engage ligands, and functional motifs in one receptor might be deduced from the identification of functional motifs in a second receptor. Testing of peptide mimetics of four NgR1 exposed loops was conducted to research their ability to antagonize the inhibitory activity of MAG, one of the key myelin ligands for the NgR1. All of the peptides were constrained by a disulfide bond, as this procedure often increases the efficacy of “loop” peptide mimetics by constraining them in a configuration that shares structural overlap with the sequence in the native protein structure (Hruby (2002) Nat. Rev. Drug Discov. 1(11):847-58; Williams et al., 2000 J. Biol Chem 275:4007-12). Three of the peptides had little or no activity; however, it remains possible that these sequences do harbor functional motifs that have been constrained in an inappropriate manner. The remaining peptide mimetic, NRL2, was an effective MAG antagonist, with near maximal inhibitory activity seen at ˜50 μg/ml (˜45 μM). The peptide had no effect on neurite outgrowth when the NgR1 complex was not activated, arguing against a trivial nonspecific effect on neurite outgrowth. Furthermore, the peptide is in effect promoting neurite outgrowth in an inhibitory environment; this would be hard to explain by a trivial mechanism. In fact, in experiments with several hundred peptides from a variety of molecules, stimulation of neurite outgrowth has not been observed as a nonspecific or trivial effect (see, e.g., Williams et al. (1994) Neuron 13(3):583-94; Williams et al. (2000) J. Biol. Chem. 275(6):4007-12; Williams et al. (2000) Mol. Cell. Neurosci. 15(5):456-64; Williams et al. (2001) J. Biol. Chem. 276(47):43879-86).

Further support for the specific nature of the antagonist properties of the NRL2 peptide has come from an examination of the structure of the sequence within the NgR1. Within the structure, two positively charged amino acids can be seen to be highly solvent exposed, and would therefore appear to be the most probable candidates for contributing to a protein-protein interaction. When two independent peptides containing these two amino acids (N-Ac-CKFRGSC-NH₂ (SEQ ID NO:32) and N-Ac-CQKFRGC-NH₂ (SEQ ID NO:33)) were made, it was found that these peptides were as effective as the longer parental peptide at inhibiting the MAG response. This demonstrates that the antagonism-of-inhibition activity of these peptides can be distilled down to a four amino acid motif (KFRG), with only two of these amino acids being optimally available for binding within the native structure. Interestingly, nerve growth factor (NGF) and a cyclized peptide from NGF that contains two positive amino acids separated by a noncharged amino acid (N-Ac-CTDIKGKEC-NH₂ (SEQ ID NO:35)) do not antagonize the inhibitory activity of myelin (data not shown).

In principle, the NRL2 peptides might inhibit NgR1 function by competing for ligand binding to the NgR1 and/or the interaction between the NgR1 and another component of the inhibitory molecule-signaling complex (e.g., p75NTR). An exclusive inhibition of MAG binding to the complex cannot explain the inhibitory activity of the peptides, as at least NRL2 was just as effective at antagonizing the inhibition induced by an antibody that binds to GT1b.

In summary, the results of this study have identified the KFRG motif in the NgR1 as a putative and/or actual binding motif. This motif, and several of the flanking amino acids (LWAWLQKFRGSSS (SEQ ID NO:36)) are fully conserved between man and rat. The 100% identity between the sequences for man and rat indicates that the antagonistic peptides disclosed herein may also be used to treat humans.

What is claimed is:

1. An antagonist to an NgR1 ligand comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

2. The antagonist as in claim 1, wherein the antagonist comprises at least one D-amino acid.

3. The antagonist of claim 1, wherein the polypeptide is cyclized.

4. The antagonist of claim 3, wherein the polypeptide is cyclized via homodetic cyclization.

5. The antagonist of claim 4, wherein the antagonist comprises at least one D-amino acid.

6. The antagonist of claim 5, wherein the polypeptide comprises the amino acid sequence of SEQ ID NO:37 or an active fragment(s) thereof.

7. The antagonist of claim 3, wherein the polypeptide is cyclized via a disulfide bond.

8. The antagonist of claim 7, wherein the polypeptide comprises an amino acid sequence selected from the group consisting of the amino acid sequence of SEQ ID NO:31, the amino acid sequence of SEQ ID NO:32, the amino acid sequence of SEQ ID NO:33, the amino acid sequence of SEQ ID NO:34, and the amino acid sequences of active fragments thereof.

9. The antagonist of claim 8, wherein the antagonist comprises at least one D-amino acid.

10. A method of screening for compounds that compete with antagonists of NgR1 ligands comprising the steps of:
(a) contacting a sample containing an NgR1 ligand and an antagonist with a compound, wherein the antagonist comprises a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof, and
(b) determining whether the interaction between the NgR1 ligand and the antagonist in the sample is decreased relative to the interaction of the NgR1 ligand and the antagonist in a sample not contacted with the compound,
wherein a decrease in the interaction of the NgR1 ligand and the antagonist in the sample contacted with the compound identifies the compound as one that competes with the antagonist.

11. The method of claim 10, wherein the compound is further identified as one that antagonizes at least one NgR1 ligand.

12. A method of antagonizing inhibition of axonal growth in a sample comprising the step of contacting the sample with an antagonist to at least one NgR1 ligand.

13. The method of claim 12, wherein the antagonist to the at least one NgR1 ligand is a peptide that mimics a functional motif of the NgR1.

14. A method of antagonizing inhibition of axonal growth in a sample comprising the step of contacting the sample with an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

15. The method of claim 14, wherein the inhibition of axonal growth is mediated by at least one NgR1 ligand.

16. The method of claim 14, wherein the antagonizing of inhibition of axonal growth results in regeneration of axons.

17. A method of antagonizing inhibition of axonal growth in a subject comprising the step of administering to the subject an effective amount of an antagonist to at least one NgR1 ligand.

18. The method of claim 17, wherein the antagonist to the at least one NgR1 ligand is a peptide that mimics a functional motif of the NgR1.

19. A method of antagonizing inhibition of axonal growth in a subject comprising the step of administering to the subject an effective amount of an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ. ID NO:18, the amino acid sequence of SEQ ID NO:22, the amino acid sequence of SEQ ID NO:37, and the amino acid sequences of active fragments thereof.

20. The method of claim 19, wherein the inhibition of axonal growth is mediated by at least one NgR1 ligand.

21. The method of claim 19, wherein the antagonizing of inhibition of axonal growth results in regeneration of axons.

22. The method of claim 19, wherein the subject has suffered an injury to the central nervous system.

23. The method of claim 22, wherein the injury is due to a stroke.

24. The method of claim 19, wherein the subject suffers from a neuronal degenerative disease.

25. The method of claim 24, wherein the neuronal degenerative disease is selected from the group consisting of multiple sclerosis, Parkinson's disease, and Alzheimer's disease.

26. A pharmaceutical composition comprising a pharmaceutically acceptable carrier and an antagonist comprising a polypeptide comprising an amino acid sequence selected from the group consisting of the amino acid sequence KFRG, the amino acid sequence GRFK, the amino acid sequence of SEQ ID NO:14, the amino acid sequence of SEQ ID NO:18, the amino acid sequence of SEQ ID NO:22, the amino acid sequence of SEQ ID NO:37, and the amino acid sequences of active fragments thereof.

27. An antagonist to an NgR1 ligand comprising a polypeptide comprising an amino acid sequence selected from the group consisting of the amino acid sequence of SEQ ID NO:2, the amino acid sequence of SEQ ID NO:4, the amino acid sequence of SEQ ID NO:6, the amino acid sequence of SEQ ID NO:10, and the amino acid sequences of active fragments thereof.

28. The antagonist of claim 27, wherein the polypeptide is cyclized.

29. The antagonist of claim 28, wherein the polypeptide is cyclized via a disulfide bond.

30. An isolated antibody capable of specifically binding to a polypeptide comprising an amino acid sequence selected from the group consisting of the amino acid sequences of SEQ ID NOs:2, 4, 6, 8, 10, 12, 14, 16, 18, 20, 22, 24, 26, 27, 28, 29, 30, 31, 32, 33, 34, 37, and the amino acid sequences of active fragments thereof.

31. The antibody of claim 30, wherein the antibody was produced in response to an immunogen comprising an antagonist to at least one NgR1 ligand.

32. An isolated antibody capable of specifically binding to an antagonist to at least one NgR1 ligand.
